(19) United States
(12) Patent Application Publication
(10) Pub. No.: US 2007/0055258 A1
(43) Pub. Date: Mar. 8, 2007

(54) Suture band

(76) Inventor: Doris Hjorth Hansen, Chiaverano (IT)

Correspondence Address: BUTZEL LONG, STONERIDGE WEST, 41000 WOODWARD AVENUE, BLOOMFIELD HILLS, MI, 48304, US

(21) Appl. No.: 10/553,143

(22) PCT Filed: Apr. 13, 2004
(86) PCT No.: PCT/IB04/01114

§ 371 Date: Oct. 13, 2006

(30) Foreign Application Priority Data
Apr. 15, 2003 (GB) 0308647.7

(51) Int. Cl. A61B 17/56

(52) U.S. Cl. 606/74

(57) ABSTRACT

The present invention provides a suture band device comprising an elongated flexible band having first and second ends, a needle attached to the first end of the band, a buckle attached proximate the second end of the band for receiving and locking the band, characterised in the that buckle comprises a locking mechanism which enables the band to be locked at any point along its length.

### FIELD OF INVENTION

The present invention relates to suture banding assembly for clamping and closing a wound or surgical incision, more particularly, for clamping and closing split bone tissue, for example, the sternum subsequent to thoracic surgery.

### BACKGROUND OF INVENTION

For proper healing of split bone tissue, for example, the split sternum, the surgically opened faces must be approximated, compressed and held together rigidly. For several bone tissue types and in particular, the sternum, this task is complicated by the physiological role played by the bone. For example, the sternum is a functional component of the thoracic cage, the incessant motion of which causes continuous stresses across the sternum. Thus, any method of closing split bone, in particular, surgically split bone, must be able to maintain compression and rigidity across the closure in the face of constant stress.

One technique used in sternum closure involves closing the sternum with a plurality of spaced stainless steel wires. Wires are placed either parasternally (around the sternum) or transternally (through the sternum) using a large, cutting needle attached to the wires. The needles are cut off and the sternal halves are brought together by twisting the wires. Finally the wires are cut short and the ends are tucked into the adjacent tissue.

While this is a useful surgical technique for closing the sternum, there are certain problems associated with this procedure. The wires are difficult to place and if to be placed transternally, the needle must be driven through the sternum, a very difficult task. The internal mammary artery is subject to being injured during the procedure. Also, the sharp wires often cause cutting of surgical gloves and may injure the surgeon. Twisting the wires while tightening may produce torsional stresses and may even severely weaken or fracture the wires. The stresses imparted by respiratory motion of the chest cage can further fatigue or break the wires. The wires may also slice through thin or osteoporotic bone. Hence, closure of the sternum with wires is a slow and tedious if not dangerous technique.

Another system employs flat flexible stainless steel bands instead of wires. Each band may be formed integrally with a surgical needle at one end and a locking member at the other end. One type of such band is disclosed in U.S. Pat. No. 5,972,006. This describes a banding assembly having a needle at one end, a long thin band, and a buckle at the other end. The buckle is provided with upstanding elements on either side of a channel which receives the band. The elements have openings adapted to be engaged by a towel clamp to retain the buckle as the band is drawn through the channel and locks into position by a locking tooth engaging an aperture on the band. Once locked into place, the band is trimmed, leaving a tail extending from the buckle which is bent and “snap fits” between the elements which are then crimped over the tail. In order to withstand the high force applied on the buckle by the towel clamp as the band is positioned, the buckle is secured to the end of the band by spaced elements which extend through openings in the band and are crimped toward each other.

There are a number of disadvantages associated with this assembly. In particular, the fit of the band around the bone is dependent on the frequency of apertures on the band. The further apart the apertures are located, the less accurate a fit is obtainable. The band tends to be too loose or too tight. The tendency of the surgeon is to overtighten the band which can cause damage to the bone and surrounding tissue. Placing the apertures closer together leads to weakening of the band and is undesirable. Furthermore, the buckle is generally of a relatively high profile and has an irregular surface due to the folding of the band back on top of the buckle and the crimping required to secure the band end. The crimping can lead to weakening of the band and the buckle, thus leading to the possibility of broken fragments of band and buckle being left in the tissue. The irregular surface of the buckle can lead to discomfort of the patient.

### SUMMARY OF INVENTION

In accordance with the present invention, there is provided a suture band device comprising an elongated flexible band having first and second ends, a needle attached to the first end of the band, a buckle attached proximate the second end of the band for receiving and locking the band, characterised in that the buckle comprises a locking mechanism which enables the band to be locked at any point along its length and in that the locking mechanism comprises a wedging means.

The present invention allows easy threading of the band through the buckle, while substantially preventing retrograde movement of the band through the buckle. Furthermore, as the band passes through the buckle, the locking mechanism maintains a substantially constant contact with said mechanism, thus allowing the band to be locked at substantially any point along its length. This avoids the necessity of an apetured band and also provides for a better fit around the bone.

The term “at any point along its length” is intended to mean any point of the band capable of engaging, in particular, capable of being threaded through the locking mechanism. Thus, for part of the band proximal the buckle, i.e. the part of the band to which the buckle is attached, the band is physically precluded from being threaded through the buckle.

The band may be manufactured from any suitable material. Preferably, a biocompatable material is used, or the material may be coated with a biocompatable material. In a preferred embodiment; relatively biologically inert metals are used, for example stainless steel to construct at least part of the device.

A strap-like elongate flexible band is preferred rather than a substantially circular wire for the reasons pointed out above. Preferably, the strap is in the range of 0.05 mm-2.0 mm thick, more preferably 0.1 mm-1.0 mm, most preferably 0.15 mm-0.3 mm thick. Most preferably, the strap has a substantially constant thickness throughout its length. Most preferably, there are no apertures present capable of engaging any part of the locking mechanism. The width of the strap is preferably in the range of 2.0 mm-1.0 cm wide, more preferably 3.0 mm-6.0 mm wide, most preferably 4.0 mm-5.0 mm wide. The length of the strap should be sufficient to encircle the bone being encircled or clamped and be capable of being threaded through the locking mechanism. The strap may have a substantially constant width and/or thickness. Alternatively, the width or thickness of the strap may be tapered towards the needle-mounted end of the strap. This enables the strap to more easily pass through the tissue. In this embodiment, the width of the strap is relatively narrow proximate the needle. The width is gradually increased, preferably constantly, until a predetermined width is obtained closer to the buckle-mounted end of the strap.

To one end of the strap there is attached a needle which is appropriately shaped in order to enable efficient penetration of tissue. In a preferred embodiment, the needle is sickle shaped. This enables the needle to “encircle” the bone more easily.

The buckle is mounted at a distal end of the strap to the needle. The buckle comprises a locking head which is removably or preferably permanently joined to the end of the strap. The locking head is adapted for receiving the other end of the strap. It is possible to provide a needle which is flattened such that it may pass through the locking head and the strap may be pulled tight to fit the encircled tissue. In this embodiment, the needle is preferably made from a substantially rigid material which makes the tissue piercing easier than using a flexible material. In a particularly preferred embodiment, the needle may be detached from the strap prior to the strap being threaded through the locking head of the buckle. This may be effected by simply cutting the needle from the end of the strap, or a part of the strap proximal the needle may be provided with a line of weakening which may be sheared when appropriate.

The locking head comprises a locking mechanism. Preferably, this is disposed entirely within the locking head. The locking head preferably has a low profile construction and is constructed from relatively smooth materials.

The locking mechanism preferably comprises a wedging means which allows entry of the strap through the locking head in one direction but prevents retrograde movement of the strap. This mechanism is preferably effected by the action of the strap on the mechanism. Preferably, the wedging means is moveable from a threading position, wherein it concurrently engages a portion of the locking head and the threaded strap, but wherein threading of the strap towards the closed position is not substantially impeded. However, friction or contact between the wedging means and the strap when a force is applied to unthread the strap from the locking head causes relative movement of the wedging means within the locking head, thereby increasing the coefficient of friction between the wedging means and the strap, thereby preventing substantial retrograde movement of the strap so as to unthread it.

The locking head preferably includes strap entry and strap exit portions, a strap-receiving aperture extending between the entry and exit portions and a floor and a roof which diverges in the direction of the exit portion. Preferably, the locking head comprises a retention means for retaining the wedging means within the locking head. The wedging means is preferably moveable between a threading position where it is adjacent the exit portion, and a locking position in where it is closer the entry portion than in the threading position and concomitantly engages the roof and the strap to wedge the strap.

Preferably the wedging means comprises a substantially spherical body or cylindrical body, preferably a ball or a roller. Preferably, the ball or roller is made from a substantially non-resilient material. Preferably, the wedging means are constructed from a relatively biologically inert metal, preferably stainless steel.

The locking head preferably has a height in the range of 2 mm-7 mm, more preferably 3 mm-5 mm. The outer surface of the locking head is preferably relatively smooth, and substantially no part of the locking mechanism is directly exposed to surrounding tissue. This minimises the discomfort felt by a patient.

Once the strap has been threaded through the locking head, the strap may be tightened around the encircled tissue until approximation of the divided tissue is achieved. The redundant length of strap that has passed through the locking head may then be trimmed away. Preferably to provide a smooth edge with little overhang from the end of the buckle.

The present invention shall now be described with reference to the drawings.

### BRIEF DESCRIPTION OF THE DRAWINGS

By way of example only, two embodiments of suture band according to the invention are described below with reference to the accompanying drawings in which:

FIG. 1 shows a perspective view of a first embodiment of a suture band, comprising a locking head, a locking ball and a strap extending from the head, encircling a fractured sternum;

FIGS. 2 and 3 show, respectively, plan and side views of the partial locking head of the first embodiment prior to attachment to the strap;

FIG. 4 shows a sectional view of the partial locking head of the first embodiment taken generally along line 4-4 of FIG. 2;

FIG. 5 shows a plan view of the strap and a hooked portion integral therewith for holding the partial locking head of the first embodiment;

FIG. 6 shows a sectional view of the strap and hooked portion of the first embodiment taken generally along line 6-6 of FIG. 5;

FIG. 7 shows a sectional view of the first embodiment showing the strap threaded into the locking head with the locking ball in its threading position;

FIG. 8, similar to FIG. 7, depicts the locking ball in its locking position;

FIG. 9 shows a perspective view of a second embodiment of a suture band, comprising a locking head, a locking ball and a strap extending from the head, encircling a fractured sternum;

FIGS. 10 and 11 show, respectively, plan and side views of the locking head of the second embodiment prior to attachment to the strap;

FIG. 12 shows a sectional view of the locking head of the second embodiment taken generally along line 12-12 of FIG. 10;

FIG. 13 shows a sectional view of the second embodiment showing the strap threaded into the locking head with the locking ball in its threading position; and

FIG. 14, similar to FIG. 13, depicts the locking ball in its locking position.

Corresponding reference characters indicate corresponding components of the present invention throughout the several views of the drawings.

### DESCRIPTION OF THE PREFERRED EMBODIMENTS

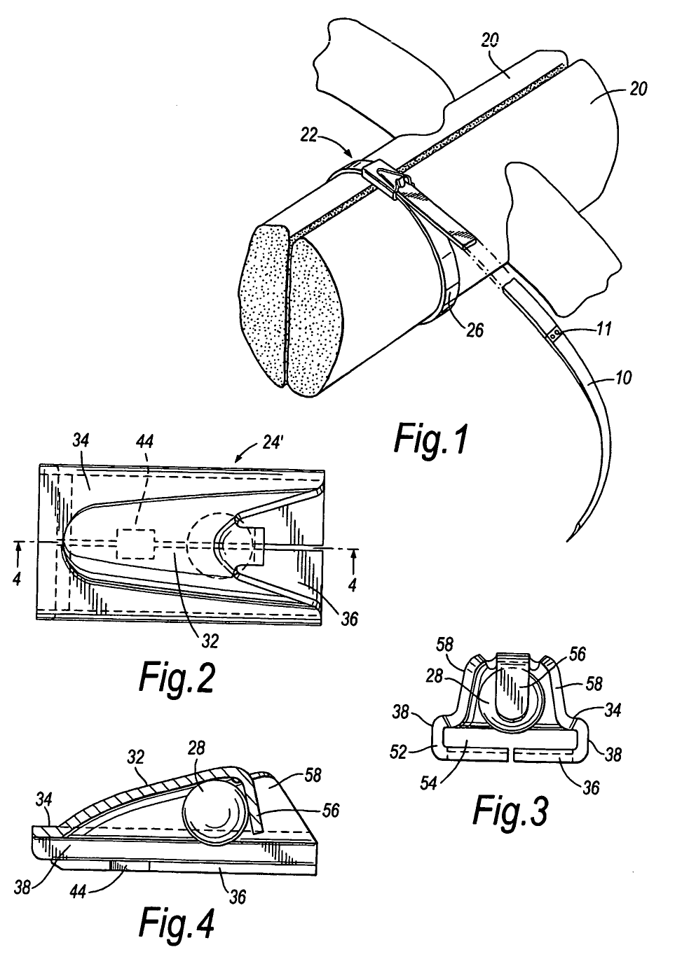

Referring now to the drawings, a first embodiment of a suture band for encircling divided bone tissue, such as the sternum 20, is generally indicated in FIG. 1 by reference character 22. The suture band is terminated at one end by a needle 10 which is attached to the end of the strap at a fixture point 11. The full length of the strap is not shown but is indicated by the hash lines in FIG. 1. Suture band 22 includes a locking head 24, an elongate strap 26 extending from the strap, and wedging means in the form of a ball or sphere 28, best shown in FIGS. 7 and 8, for retaining the strap within the locking head. Preferably the ball, head and strap are formed of stainless steel to give the suture band high strength and excellent resistance to corrosion. The ball 28 preferably has a roughened surface to ensure that it wedges securely.

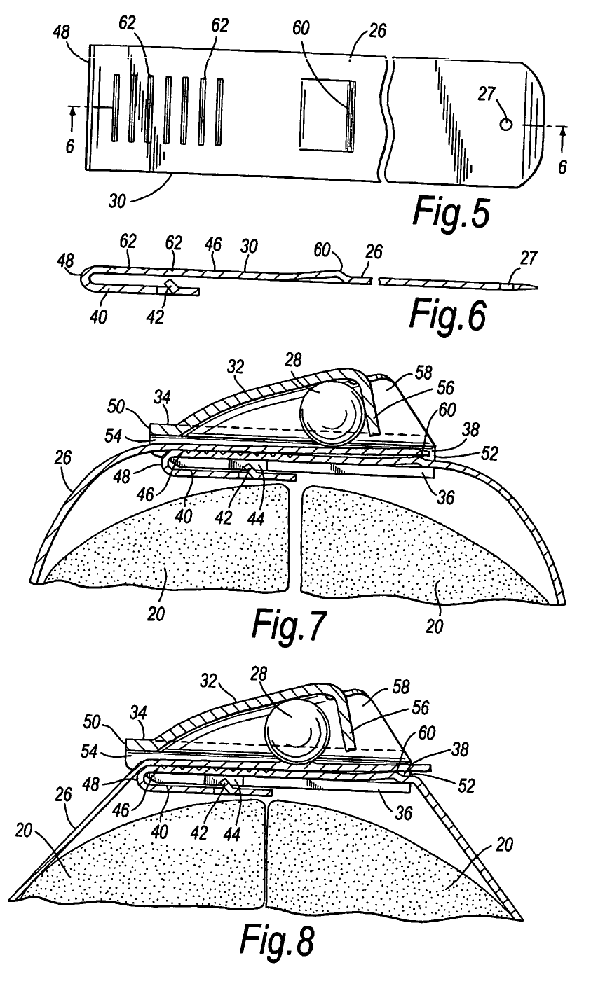

The suture band of the present invention is formed by assembling a partial locking head 24′, shown in FIGS. 2-4, and the strap 26 and a hooked portion 30 formed integral with the strap as shown in FIGS. 5 and 6. After assembly, the hooked portion is a component on the completed locking head 24. Referring to FIGS. 2-4, partial locking head 24′ comprises a roof 32, a ceiling 34 and a bottom wall 36 with the ceiling and bottom wall joined by a pair of sidewalls 38. Hooked portion 30 includes a distal end 40 having a resilient latching finger 42 for reception in a window 44 formed in bottom wall 36. Thus finger 42 and window 44 constitute latching means for holding the head and hooked portion together. Hooked portion 30 also comprises a floor 46 of the assembled locking head 24 with floor 46 and distal end 40 extending generally parallel to one another, joined by a bight 48 and spaced sufficiently to receive the bottom wall 36 therebetween. Strap 26 is shown unattached to a needle. However, the attachment point 27 is suitable for attachment of a needle. This may be done by splitting a needle and attaching it to the strap at at least two points. A needle has two ends: a blunt end and a sharp end. To attach the needle to the strap, the blunt end is split, the strap end adjacent to attachment point 27 is placed between the two parts formed by splitting the blunt end and then the needle is fixed to the strap.

As shown in FIGS. 7 and 8, locking head 24 includes a strap entry face 50, a strap exit face 52 and a strap-receiving aperture 54 extending therebetween. Roof 32 and floor 46 diverge in the direction of exit face 52. Ball 28, which has a textured or roughened surface to increase its coefficient of friction with the strap, is captively held between the roof and floor by retention means comprising a finger 56 extending from the roof toward the floor adjacent exit face 52. One end of roof 32 joins ceiling 34 adjacent entry face 50 with spaced side wings 58 joining other parts of the roof and ceiling. Portions of side wings 58 adjacent exit face 52 serve as a reaction surface for the nose of a strap tightening tool.

Locking ball 28 is movable between a threading position, shown in FIG. 7, wherein ball 28 is disposed engaging finger 56 adjacent exit face 52 and a locking position, shown in FIG. 8, wherein the ball is closer entry face 50 and securely engages the threaded strap. It is noted that with ball 28 in its threading position and concurrently engaging finger 56 and roof 32, the spacing between ball 28 and floor 46 is greater than the thickness of the strap. To insure that the locking ball is in continuous engagement with the threaded strap without regard to the position of the ball or the orientation of the locking head, tie 22 comprises deflection means functioning along with ball 28 to effect a bend in the threaded portion of the strap.

The deflection means comprises a raised portion or protuberance 60 for deflecting the threaded strap away from floor 46 as the threaded strap exits the locking head. Protuberance 60 is disposed adjacent strap exit face 52 either extending from floor 46, as is shown in the drawings, or extending from the strap. As shown in FIG. 7, as the strap is threaded through the locking head it is engaged at three spaced locations causing the strap to bend and remain in engagement with the locking ball 28. More specifically, the strap is engaged by floor 46 adjacent strap entry face 50, by locking ball 28 and by protuberance 60. Preferably, floor 46 has a series of regularly spaced transverse grooves 62 of triangular configuration for biting into the locked strap to further resist the application of strap withdrawal force. Also preferably the roof and floor diverge at approximately ten degrees.

The distance between the threading position and locking position of the ball is preferably minimised to less than 1.5 mm, most preferably less than 1.0 mm.

Operation of suture 22 is as follows: After strap 26 is deformed to encompass the bone tissue to be held, the needle is sheared away to leave a tip of the strap. The tip is inserted into locking head 24. Continued threading of the strap causes the strap to bend resulting in positive locking of the strap and ball no matter at what angle the head is held. Release of the tightened strap causes the locking ball to move to its locking position, shown in FIG. 8, where the strap is compressively held between the ball and the floor resulting in the ball indenting the threaded strap. It will be appreciated that if the suture band encircles resilient material such as bone or soft tissue and the excess portion of the threaded strap is severed adjacent the strap exit face by a strap tightening tool, the severed end of the strap will withdraw slightly inside the locking head to avoid exposed sharp edges. Also it should be appreciated that no portion of the locking mechanism extends from the locking head. In certain prior art roller locking straps, a portion of the roller locking means extended from the head where it might be inadvertently engaged causing movement of the roller locking means resulting in release or loosening of the strap.

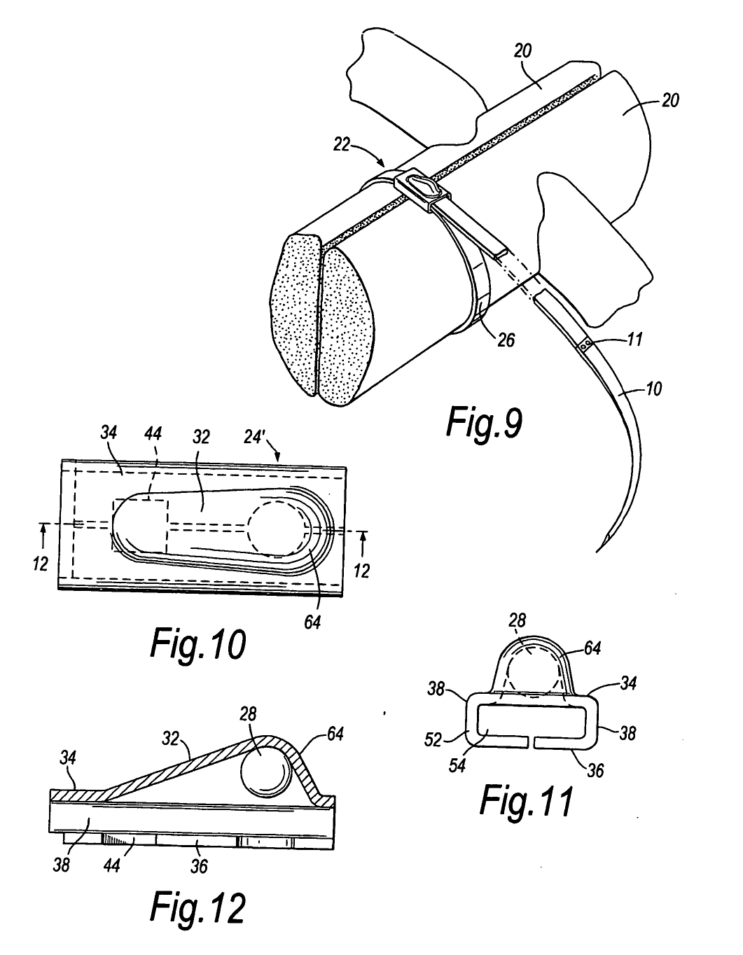

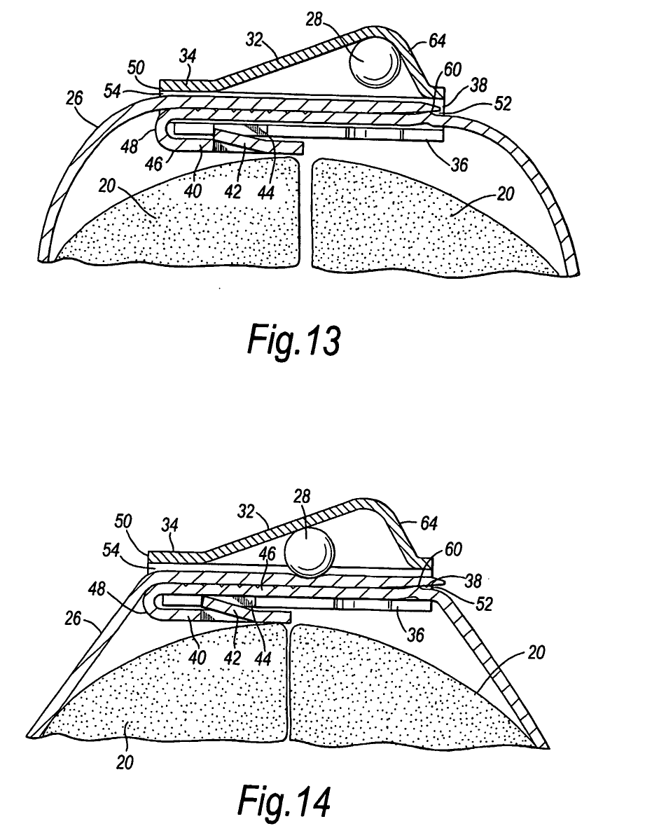

The second embodiment of the suture band according to the invention is shown in FIGS. 9 to 14. The hooked portion 30 and strap 26 used in this embodiment are identical to the hooked portion 30 and strap 26 used in the first embodiment. The partial locking head 24′ differs in that the roof presents a smooth upper surface 64 which lacks side walls 58 and engaging finger 56. The advantage of this arrangement is that the locking head is more comfortable for the user as it lacks sharp edges. The suture band according to the second embodiment of the invention works in the same way as the first embodiment.

1. A suture band device comprising an elongated flexible band having first and second ends, a needle attached to the first end of the band, a buckle attached proximate the second end of the band for receiving and locking the band, characterised in that the buckle comprises a locking mechanism which enables the band to be locked at any point along its length and in that the locking mechanism comprises a wedging means.

2. A device according to claim 1, wherein the locking mechanism substantially prevents retrograde movement of the band through the buckle.

3. A device according to claim 1 or claim 2, wherein the wedging means is selected from a substantially spherical or cylindrical body.

4. A device according to claim 3 wherein the body has a roughened surface.

5. A device according to any one of the preceding claims, wherein the band maintains substantially constant contact with the locking mechanism during threading.

6. A device according to any one of the preceding claims, wherein the locking mechanism has a substantially smooth upper surface.

7. A method of approximating bone tissue comprising encircling said tissue with a suture band device as defined in any one of the preceding claims, threading a buckle of the suture banding device with a band of the suture banding device, and constricting the device around the bone tissue.
